(19) United States
(12) Patent Application Publication
(10) Pub. No.: US 2020/0281839 A1
(43) Pub. Date: Sep. 10, 2020

(54) METHOD FOR TREATING KERATINOUS MATERIALS USING AMINATED AND OXIDISED POLYSACCHARIDE

(71) Applicant: L'Oreal, Paris (FR)

(72) Inventors: Michel PHILIPPE, Aulnay-Sous-Bois (FR); Gwenaëlle JEGOU, Aulnay-Sous-Bois (FR)

(21) Appl. No.: 16/331,378

(22) PCT Filed: Sep. 12, 2017
(86) PCT No.: PCT/EP2017/072828

§ 371 Date: May 21, 2020

(30) Foreign Application Priority Data
Sep. 12, 2016 (FR) 1658478

(51) Int. Cl. A61K 8/73; A61Q 5/00

(52) U.S. Cl. CPC A61K 8/73; A61Q 5/00

(57) ABSTRACT

The invention concerns i) a method for treating keratinous materials, in particular human keratinous fibres such as hair, implementing a) at least one aminated and oxidised polysaccharide comprising at least one carboxy(late) group, in particular at least one aminated and oxidised inulin with one or more carboxy or carboxylate group(s); ii) a composition comprising at least the ingredient a); iii) the use of at least a) for treating keratinous fibres, in particular human keratinous fibres such as hair, and iv) a kit or device with several compartments comprising at least one ingredient a). The invention helps obtain good cosmetic conditioning properties in terms of conditioning keratinous materials, in particular hair, with a long-lasting effect.

The invention relates to i) a process for treating keratin materials, in particular keratin fibers, more particularly human keratin fibers such as the hair, employing a) at least one oxidized amino polysaccharide comprising at least one carboxy(late) group, in particular at least one oxidized amino inulin comprising carboxy or carboxylate group(s); ii) a composition comprising at least the ingredient a); iii) the use of at least one ingredient a) for treating keratin fibers, in particular human keratin fibers such as the hair; and iv) a multi-compartment kit or device comprising at least one ingredient a).

Hair is generally damaged and weakened by the action of external atmospheric agents such as light, sun and bad weather, and also by mechanical or chemical treatments, such as brushing, combing, dyeing, bleaching, permanent-waving, relaxing and repeated washing. Hair is thus damaged by these various factors and may in the long run become dry, coarse, brittle or dull or split or limp.

Thus, to overcome these drawbacks, it is common practice to resort to hair treatments which make use of compositions intended for conditioning the hair appropriately by giving it satisfactory cosmetic properties, especially smoothness, sheen, a soft feel (a natural feel; the hair is no longer coarse), suppleness, a lightweight feel, good disentangling properties leading to easy combing, and good manageability of the hair which is thus easy to shape.

These hair care compositions can, for example, be conditioning shampoos, hair conditioners, masks or serums. However, the conditioning effect obtained fades out in the course of successive shampoo washes and does not show satisfactory persistence on shampooing.

It is known practice to employ care compositions comprising reducing sugars such as monosaccharides, used as conditioning agents, especially to repair keratin fibers which have been damaged by harsh treatments.

Indeed, patent application US 2002/0193264 describes a process for conditioning keratin fibers, in which at least one sugar chosen from monosaccharides is applied to said fibers, and a step of heating the keratin fibers is carried out. Similarly, patent application US 2002/0172653 discloses a process for conditioning keratin fibers comprising a step of applying to said fibers a sugar chosen from specific C₅-C₇ monosaccharides and a step of heating the keratin fibers. However, the use of reducing sugars followed by a heat treatment may lead to an undesired modification of the color of the keratin fibers. Furthermore, reducing sugars degrade easily, especially under the action of shampoos, which results in the cosmetic properties conferred on the fibers not persisting. Thus, the keratin fibers are not treated in a long-lasting manner, in particular not protected, repaired or cosmetically transformed.

In the field of dyeing, patent application FR 2 944 967 discloses the use of oxidized polysaccharides for protecting the color of keratin fibers that have been artificially dyed. International application WO 2013/132062 also discloses a process for treating the hair, which consists in using one or more oxidized polysaccharides and in raising the temperature of the keratin fibers. In addition, patent application US 2003/0053977 describes a process for protecting keratin fibers which uses heat and a glucosamine.

Nonetheless, the results obtained are not always satisfactory in terms of manageability, repair and/or protection of the keratin fibers, in particular damaged and/or sensitized hair, in terms of softness of surfaces of said fibers, in particular at the dry ends, or are not always satisfactory for disentangling wet or damp keratin fibers.

Moreover, new reactive materials that can be used in cosmetics and that are obtained by means of environmentally friendly processes are sought. In particular, it is valuable to have available new multi-reactive polymers capable of binding in a long-lasting manner to keratin substrates in such a way as to provide these substrates with a long-lasting benefit. Indeed, there are no aminated green materials of natural origin which have other oxidized reactive functions.

There is therefore a real need to develop a composition and a process for treating keratin fibers such as the hair, which are capable of treating in a long-lasting manner, in particular conditioning, making manageable and/or protecting the hair, without leading to a modification of the color thereof. It is also advantageous to find a means for treating damaged keratin fibers by repairing them, that is to say by intrinsically improving the cosmetics of the keratin fibers, reducing breakage of the keratin fibers and/or preventing breakage of the keratin fibers.

This (these) technical problem(s) is (are) solved by the process for treating keratin materials, especially keratin fibers, in particular human keratin fibers, preferably the hair, comprising a step consisting in applying, to said fibers, a) one or more oxidized amino polysaccharide(s) comprising at least one carboxy(late) group, said polysaccharide(s) preferably being nonionic or anionic.

A subject of the invention is also a composition comprising one or more oxidized amino polysaccharide(s) comprising at least one carboxy (late) group, said polysaccharide(s) preferably being nonionic or anionic.

Another subject of the invention is the use of the ingredients a) for improving the manageability, the conditioning of keratin fibers, for repairing damaged keratin fibers and/or for preventing breakage of keratin fibers.

In particular, hair treated with the process according to the invention remains well-behaved since the presence of frizziness is not observed. Thus, the hairs are aligned, smooth and disentangle easily, which makes them easier to comb. The treated hair also has more body (it is not limp) and is thus easier to style. The treated hair shapes well. Moreover, the treated hair is also shinier and feels softer. It is stronger and less brittle.

After treatment, the hair is not laden, and has a natural feel.

The process according to the invention has the advantage of giving good persistence of these good hair-conditioning cosmetic properties after shampooing. Thus, the treated hair is conditioned in a long-lasting manner.

In the following text, unless indicated otherwise:
the term “organic or mineral acid salt” is intended to mean cosmetically acceptable organic or mineral acid salts, more particularly the salts chosen from a salt derived from i) hydrochloric acid HCl, ii) hydrobromic acid HBr, iii) sulfuric acid H₂SO₄, iv) alkylsulfonic acids: Alk-S(O)₂OH such as methylsulfonic acid and ethylsulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)OH; xiv) triflic acid CF₃SO₃H; and xv) tetrafluoroboric acid HBF₄;
the term “cationic counterion” is intended to mean cosmetically acceptable cationic counterions, particularly chosen from cations of alkali metals or alkaline-earth metals such as sodium, potassium or calcium, ammonium R,R′,R″N⁺—, or phosphonium R,R′,R″P⁺—; with R, R′ and R″, which may be identical or different, representing a hydrogen atom, or a (C₁-C₆)alkyl group, optionally substituted in particular with a hydroxyl group, such as hydroxyethyl;
the term “alkyl” is intended to mean a linear or branched radical containing from 1 to 14 carbon atoms, in particular from 1 to 8 carbon atoms, preferably from 1 to 6 carbon atoms, more preferentially from 1 to 4 carbon atoms, for example methyl, ethyl, n-propyl, isopropyl, butyl, n-pentyl, n-hexyl, preferably methyl;
the term “alkoxy” is intended to mean an alkyl-oxy group with “alkyl” as defined previously;
the term “optionally substituted” followed by (C₁-C₆) alkyl is intended to mean that said alkyl group can be substituted with one or more groups, which may be identical or different, chosen from i) hydroxyl, ii) halogen, iii) (C₁-C₄)alkoxy, iv) hydroxy(C₂-C₄)alkoxy; v) (di)(hydroxy(C₁-C₄)(alkyl)amino, vi) Rₐ—Zₐ—C(Z_{b})-Z_{c}—, and v) Rₐ—Zₐ—S(O)ₜ—Z_{c}— with Zₐ and Z_{b}, which may be identical or different, representing an oxygen or sulfur atom or a group NRₐ′, Z_{c} representing a bond, an oxygen or sulfur atom or a group NRₐ; Rₐ representing a hydrogen atom or a (C₁-C₄)alkyl group and R_{a′} representing a hydrogen atom or an alkyl group and t is 1 or 2;
an “aryl” radical represents a monocyclic or polycyclic fused or non-fused carbon-based group, comprising from 6 to 22 carbon atoms, at least one ring of which is aromatic; in particular, the aryl radical is a phenyl, biphenyl, naphthyl, indenyl, anthracenyl or tetrahydronaphthyl, preferably phenyl;
a “heteroaryl radical” represents a 5- to 22-membered, monocyclic or polycyclic, fused or non-fused group, comprising from 1 to 6 heteroatoms chosen from nitrogen, oxygen and sulfur atoms, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridinyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl;
moreover, the addition salts that may be used in the context of the invention are especially chosen from addition salts with a cosmetically acceptable base such as basifying agents as defined below, for instance alkali metal hydroxides, such as sodium hydroxide or potassium hydroxide, aqueous ammonia, amines or alkanolamines;
the term “(hetero)cyclo(C₅-C₇)alkyl” is intended to mean a monocyclic or bicyclic, preferably monocyclic, fused or non-fused group which is saturated or contains one or more ethylenic unsaturations, comprising from 5 to 7 ring members, comprising in at least one ring a heteroatom N, possibly containing from 1 to 3 additional non-adjacent heteroatoms chosen from a nitrogen, oxygen and sulfur atom, this heterocycle possibly being substituted with one or more radicals, which may be identical or different, chosen from alkyl, hydroxyalkyl and alkoxy radicals; preferentially, according to the present invention, the saturated or unsaturated, preferably saturated, 5- to 8-membered heterocycle is chosen from piperidyl, pyrrolidinyl, piperazinyl and morpholinyl;
the “aryl” or “heteroaryl” radicals or the aryl or heteroaryl part of a radical may be substituted with at least one substituent borne by a carbon atom, chosen from:
   an optionally substituted C₁-C₆ alkyl radical;
   a halogen atom;
   a hydroxyl group;
   a C₁-C₂ alkoxy radical;
   a C₂-C₄ (poly)hydroxyalkoxy radical;
   an amino radical;
   a 5- or 6-membered heterocycloalkyl radical;
   a 5- or 6-membered heteroaryl radical, optionally substituted with a (C₁-C₄) alkyl radical, preferentially methyl;
   an amino radical substituted with one or two identical or different C₁-C₆ alkyl radicals, optionally bearing at least:
      i) a hydroxyl group,
      ii) an amino group optionally substituted with one or two optionally substituted C₁-C₃ alkyl radicals, said alkyl radicals possibly forming with the nitrogen atom to which they are attached a saturated or unsaturated, optionally substituted 5- to 7-membered heterocycle, optionally comprising at least one other nitrogen or non-nitrogen heteroatom,
   an acylamino radical (—NR—C(O)—R′) in which the radical R is a hydrogen atom or a C₁-C₄ alkyl radical optionally bearing at least one hydroxyl group and the radical R′ is a C₁-C₂ alkyl radical;
   a carbamoyl radical ((R)₂N—C(O)—) in which the radicals R, which may be identical or different, represent a hydrogen atom or a C₁-C₄ alkyl radical optionally bearing at least one hydroxyl group;
   an alkylsulfonylamino radical (R′—S(O)₂—N(R)—) in which the radical R represents a hydrogen atom or a C₁-C₄ alkyl radical optionally bearing at least one hydroxyl group and the radical R′ represents a C₁-C₄ alkyl radical or a phenyl radical; an aminosulfonyl radical ((R)₂N—S(O)₂—) in which the radicals R, which may be identical or different, represent a hydrogen atom or a C₁-C₄ alkyl radical optionally bearing at least one hydroxyl group;
   a carboxylic radical in acid or salified (preferably with an alkali metal or a substituted or unsubstituted ammonium) form;
   a cyano group;
   a nitro or nitroso group;
   a polyhaloalkyl group, preferentially trifluoromethyl;
the term “optionally substituted C₁-C₆ alkyl” is intended to mean that the alkyl may be substituted with one or more radicals chosen from the following radicals: hydroxyl, C₁-C₂ alkoxy, C₂-C₄ (poly)hydroxyalkoxy, acylamino, amino substituted with two identical or different C₁-C₄ alkyl radicals optionally bearing at least one hydroxyl group or the two radicals possibly forming, with the nitrogen atom to which they are attached, a saturated or unsaturated, optionally substituted, 5- to 7-membered and preferably 5- or 6-membered heterocycle optionally comprising another nitrogen or non-nitrogen heteroatom;
the expression “at least one” is equivalent to “one or more”; and
the expression “inclusive” for a range of concentrations means that the limits of the range are included in the defined interval.

### a) OXIDIZED AMINO POLYSACCHARIDES COMPRISING CARBOXYL(LATE) GROUPE(S)

The polysaccharide(s) of the invention are amino polysaccharides, that is to say that they comprise at least one amino group. the term “amino group(s)” is intended to mean that the polysaccharide is substituted with one or more amino groups NR₁R₂, i.e. at least one of the hydroxyl groups of the saccharide unit is replaced with a group NR₁R₂ with R₁ and R₂, which may be identical or different, representing i) a hydrogen atom, ii) a (C₁-C₁₄)alkyl group that is optionally substituted, preferably with one or more hydroxyl or NH₂ groups, iii) a (hetero)aryl group such as phenyl, iv) a (hetero)aryl(C₁-C₁₄)alkyl group such as benzyl, v) a (hetero)cyclo(C₅-C₇)alkyl group such as cyclohexyl, morpholinyl, piperazinyl, piperidinyl, vi) a (hetero)cyclo(C₅-C₇)alkyl(C₁-C₁₄)alkyl group such as cyclohexylmethyl, vii) —C(Y)—(Y′)_{q}-R′₁ with Y and Y′, which may be identical or different, representing an oxygen atom, a sulfur atom or N(R′₂), preferably oxygen, q=0 or 1, preferably 0; and R′₁ and R′₂ representing i) to vi) of R₁ and R₂ defined previously. Preferably, R₁ and R₂ represent a hydrogen atom.

In addition, the polysaccharide(s) of the invention are oxidized and they comprise at least one carboxy(late) group; this implies that they comprise at least one carboxy group —C(O)—OH or carboxylate group —C(O)O⁻, M⁺ with M⁺ representing a cationic counterion. Preferably, M⁺ represents an alkali metal or an alkaline-earth metal such as Na⁺ or K⁺ or an ammonium R,R′,R″N⁺— with R, R′ and R″, which may be identical or different, representing a hydrogen atom, or a (C₁-C₆)alkyl group optionally substituted in particular with a hydroxyl group such as hydroxyethyl.

According to one particular embodiment of the invention, the oxidized amino polysaccharide(s) comprising at least one carboxylate or carboxylic acid group is (are) anionic or nonionic.

The term “polysaccharides” is intended to mean glycans, polyglycosides, polysaccharides or complex carbohydrates, which are polymers constituted of several monosaccharides bonded together by O-glycosidic bonds. They are more particularly polymers formed from a certain number of monosaccharides having the general formula: —[Cₓ(H₂O)_{y})]ₙ— where x is an integer greater than or equal to 5, preferably x is greater than or equal to 6, in particular x is between 5 and 7 inclusive and preferably x=6, and y is an integer which represents x−1, and n is an integer greater than or equal to 2, particularly of between 3 and 1000 inclusive, more particularly between 5 and 500 and preferentially between 10 and 200.

According to one particular embodiment of the invention, the anionic or nonionic oxidized amino polysaccharides comprising carboxy(late) group(s) are constituted of monosaccharide units which may comprise five carbon atoms or more, preferably six carbon atoms or more, and more particularly six carbon atoms.

The nonionic or anionic oxidized amino polysaccharides also comprise one or more aldehydes and optionally one or more anionic groups.

The oxidized amino polysaccharides comprising at least one carboxy(late) group, according to the invention, are in particular chosen from those of formula (I) below:

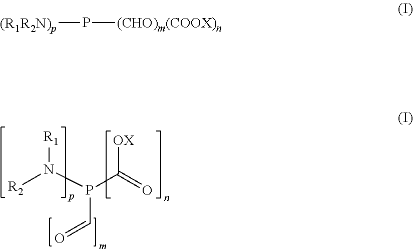

Corresponding to formula (I) wherein:
P represents a polysaccharide chain constituted of monosaccharides comprising 5 carbon atoms or more than 5 carbon atoms, preferably 6 or more than 6 carbon atoms and more particularly 6 carbon atoms;
R₁ and R₂, which may be identical or different, are as defined previously, preferably R₁ and R₂ are chosen from a hydrogen atom and —C(O)—R′₁ representing i) to vi) defined previously; preferably R₁ and R₂ represent i) a hydrogen atom, ii) a (C₁-C₆)alkyl group such as methyl, or a (C₁-C₆)alkylcarbonyl group such as acetyl;
X represents hydrogen atom, a cationic counterion, in particular chosen from i) alkali metal, ii) alkaline earth metal, such as sodium or potassium, iii) ammonium R,R′,R″N⁺with R, R′ and R″ as defined previously, and iv) cationic counterions derived from organic bases, such as aqueous ammonia, organic amines such as monoethanolamine, diethanolamine, triethanolamine and 3-amino-1,2-propanediol, and basic amino acids such as lysine, arginine, sarcosine, ornithine and citrulline;
m+n is greater than or equal to 1;
m is such that the degree of substitution of the polysaccharide with one or more aldehyde groups (DS(CHO)), which is greater than or equal to 0, in particular m is within the range of from 0 to 2, more particularly ranging from 0.001 to 1.8, preferably ranging from 0.005 to 1.5;
n is such that the degree of substitution of the polysaccharide with one or more carboxylic groups (DS(COOX)) is greater than 0, in particular less than or equal to 2, preferably between 0.001 and 1.5, inclusive;
p is such that the degree of substitution of the polysaccharide with one or more amine groups (DS(R₁R₂N)) is greater than 0, in particular less than or equal to 2, preferably between 0.001 and 1.5, inclusive.

The expression “degree of substitution DS(CHO) or DS(COOX) of the polysaccharides according to the invention” is intended to mean the ratio between the number of carbons oxidized to give an aldehyde or carboxylic group for all the repeating units and the number of elementary monosaccharides (even opened by preoxidation) constituting the polysaccharide.

The expression “degree of substitution (DS(R₁R₂N)) of the polysaccharides according to the invention” is intended to mean the ratio between the number of hydroxyl groups substituted with an amine group NR₁R₂ for all the repeating units and the number of elementary (even open) monosaccharides constituting the polysaccharide.

The aldehyde CHO and carboxy(late) COOX groups may be obtained during the oxidation of certain carbon atoms, for example in position C2, C3 or C6, of a saccharide unit comprising 6 carbon atoms. Preferably, the oxidation takes place at C2 and at C3, more particularly from 0.01% to 75% by number and preferably from 0.1% to 50% by number of the rings having possibly been opened.

The amine groups R₁R₂N can be obtained during the reductive amination of certain hydroxyl groups borne by the carbon atoms, for example in position C2, C3 or C6, of a saccharide unit comprising 6 carbon atoms; preferably, the reductive amination takes place at C2 and at C3, more particularly at C2 from 0.01% to 75% by number and preferably from 0.1% to 50% by number of the rings possibly having been opened.

The polysaccharide chain, represented by P in formula (I) above, is preferably chosen from inulins, celluloses, starches, guar gums, xanthan gums, pullulan gums, alginate gums, agar-agar gums, carrageenan gums, gellan gums, chitosan, gums arabic, xyloses and tragacanth gums, and derivatives thereof, cellobiose, maltodextrin, scleroglucan, chitosan, ulvan, fucoidan, alginate, pectin, heparin and hyaluronic acid, or mixtures thereof.

According to one particular embodiment of the invention, the polysaccharide chain, the oxidized amino polysaccharide(s) comprising at least one carboxy(late) group, according to the invention, are chosen from those of formula (I) as defined previously wherein P corresponding to the polysaccharide chain is chosen from celluloses, (C₁-C₆)alkylcelluloses, hydroxy(C₁-C₆)alkylcelluloses such as hydroxyethylcelluloses and hydroxypropylcelluloses, carboxycelluloses, carboxy(C₁-C₆)alkylcelluloses such as carboxymethylcelluloses, starches, inulins, and preferably inulin.

More preferentially, the polysaccharide chain is chosen from inulins and starches.

Even more preferentially, the polysaccharide chain is inulin.

The term “derivative” is intended to mean the compounds obtained by chemical modification of the mentioned compounds. They may be esters, amides or ethers of said compounds.

The oxidation may take place according to a process known in the art, for example according to the process described in FR 2 842 200, in document FR 2 854 161 or in the article “Hydrophobic films from maize bran hemicelluloses” by E. Fredon et al., Carbohydrate Polymers 49, 2002, pages 1 to 12. Another oxidation process is described in the article “Water soluble oxidized starches by peroxide reaction extrusion” Industrial Crops and Products 75 (1997) 45-52—R. E. Wing, J. L. Willet. These oxidation processes are easy to perform, are efficient and do not generate any toxic by-products or by-products that are difficult to remove.

The peroxides that may be used in these oxidation processes may be an alkali metal or alkaline-earth metal percarbonate or perborate, an alkyl peroxide, peracetic acid or hydrogen peroxide. Hydrogen peroxide is particularly preferred, in so far as it is readily accessible and does not produce interfering by-products.

The amount of peroxide in the reaction medium is between 0.05 and 1 molar equivalent per glucose unit of the polysaccharide, preferably between 0.1 and 0.8 molar equivalent. It is preferable to add the peroxide in successive portions, leaving the reaction medium stirring between two additions.

A single phthalocyanin or a mixture of phthalocyanins, for example a mixture of cobalt phthalocyanin and of iron phthalocyanin, may be used as catalyst in the oxidation process. The amount of catalyst depends on the desired degree of substitution. In general, a small amount, for example an amount corresponding to 0.003 to 0.016 molar equivalent per 100 glucose units of polysaccharide, is suitable.

The process may also be performed by placing the polysaccharide in pulverulent form in contact with the catalyst dissolved in a small volume of water and with the peroxide. This process is referred to as a “semi-dry” process.

The process may be carried out by reactive extrusion in the presence of peroxide.

For the reductive amination, it is preferred to use, firstly, a polysaccharide oxidized beforehand according to the techniques known to those skilled in the art as previously mentioned, then to react it with an organic amino base R₁R₂N—R₃ with R₁ and R₂ as defined previously and R₃ representing a hydrogen atom, more preferentially R₁R₂N—R₃ representing NH₃, in particular aqueous NH₃ or aqueous ammonia. The reaction is followed by a reduction step which can be carried out with a catalyst such as Ni, or Pd on graphite in the presence of H₂ under pressure (such as 10 bar) (see for example U.S. Pat. No. 6,596,861 or Chem. Eur. J. 1130-1144 (2007); ibid 10196-10200 (2008)).

Preferentially in the process for preparing the polysaccharide(s) a) of the invention, the polysaccharide is first oxidized as described previously, followed by one or more successive reductive aminations.

More preferentially, the polysaccharide(s) a) according to the invention are obtained by oxidation and reductive amination:
of inulin,
of celluloses, in particular i) cellulose, ii) carboxy(C₁-C₆)alkylcelluloses such as carboxymethylcellulose, iii) (hydroxy)(C₁-C₆)alkylcelluloses such as methylcellulose, hydroxyethylcellulose, hydroxypropylcellulose or hydroxypropylmethylcellulose,
of starches, in particular i) starch, ii) starch acetates, iii) (hydroxy)C₁-C₆)alkyl starch, such as hydroxyethyl starch or hydroxypropyl starch,
of guar gums, carboxy(C₁-C₆)alkylguar gums such as carboxymethylguar, carboxy(C₁-C₆)alkylhydroxy(C₁-C₆)alkylguar gums such as carboxymethylhydroxypropylguar gums, hydroxy(C₁-C₆)alkylguar gums, hydroxyethylguar gums, hydroxypropylguar gums,
of xyloses or xanthan gums, or carrageenan gums, of cellobiose, of maltodextrin, of scleroglucan, of chitosan, of ulvan, of fucoidan, of alginate, of pectin, of heparin and of hyaluronic acid,
of cyclodextrin and derivatives thereof,
or mixtures thereof.

Preferentially, the polysaccharide is obtained by oxidation and reductive amination of inulin or of starch, more preferentially by oxidation and reductive amination of inulin.

Preferentially, the polysaccharide is obtained by oxidation and reductive amination of inulin.

According to one embodiment, the polysaccharide is obtained by oxidation of inulin by performing a reactive extrusion process in the presence of hydrogen peroxide.

The polysaccharide chain before and after oxidation and reductive amination preferably has a weight-average molecular weight ranging from 400 to 15 000 000, better still from 500 to 10 000 000 and more particularly from 500 to 50 000 g/mol.

The polysaccharides that are most particularly preferred in the invention are those corresponding to formula (I) wherein: P represents a polymer chain derived from inulin or from starch, preferably from inulin; m is such that the degree of substitution of the polysaccharide with one or more aldehyde groups (DS(CHO)) is greater than or equal to 0 less than 2.5, n is such that the degree of substitution of the polysaccharide with one or more carboxylate groups (DS(COOX)) is within the range of from 0.001 to 2, and p is such that the degree of substitution of the polysaccharide with one or more amine groups (DS(R₁R₂N)) is between 0.001 and 2, inclusive.

Even more preferably, the P radical of formula (I) as defined previously represents a polymer chain derived from inulin, m is within the range of from 0 to 1, n and p, which may be identical or different, are within the range of from 0.01 to 2.

Advantageously, the oxidized amino polysaccharide(s) as defined above is (are) in a content ranging from 0.05% to 15% by weight, preferably ranging from 0.1% to 10% by weight and more preferentially ranging from 0.2% to 6% by weight relative to the total weight of the composition.

### The Compositions

The composition of the invention comprises one or more oxidized amino polysaccharide(s) comprising at least one carboxy(late) group as defined previously.

The composition(s) of the invention are cosmetic, i.e. they contain a physiologically acceptable medium, that is to say a medium that is compatible with human keratin materials such as the skin (of the body, face, around the eyes or the scalp), the hair, the eyelashes, the eyebrows, bodily hair, the nails or the lips.

The physiologically acceptable medium of the composition(s) used in the process according to the invention is advantageously an aqueous medium. It may be constituted, for example, of water or of a mixture of water and of at least one cosmetically acceptable organic solvent. Examples of organic solvents that may be mentioned include C₂-C₄ lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether and diethylene glycol monomethyl ether or monoethyl ether; and mixtures thereof.

Preferably, the cosmetic composition comprises from 50 to 99.5% by weight of water, relative to the weight of the composition.

The composition used according to the invention may also contain one or more cosmetic additives chosen from nonionic, anionic, cationic and amphoteric surfactants, vitamins and provitamins, including panthenol, sunscreens, fillers, colorants, nacreous agents, opacifiers, sequestrants, film-forming polymers, cationic, anionic or neutral polymers other than the polysaccharides a) according to the invention, associative polymers, plasticizers, thickeners, oils, antioxidants, antifoams, moisturizers, emollients, penetrants, fragrances and preservatives; preferably one or more nonionic, anionic, cationic or amphoteric surfactants, cationic, anionic or neutral polymers other than the polysaccharides a) according to the invention, or associative polymers.

The composition used according to the invention may be in any galenical form conventionally used for application to the hair and especially in the form of aqueous solutions, aqueous-alcoholic solutions, oil-in-water (O/W), water-in-oil (W/O) or multiple (triple: W/O/W or O/W/O) emulsions, aqueous gels or aqueous-alcoholic gels. These compositions are prepared according to the usual methods. Preferably, the composition is in the form of an aqueous or aqueous-alcoholic solution or gel.

### pH of the Composition(s):

According to one particular embodiment of the invention, the composition which comprises a) one or more oxidized amino polysaccharide(s) comprising at least one carboxy(late) group as defined previously, in particular inulin, is at a pH of between 2.5 and 9.5, inclusive.

According to one preferred embodiment of the invention, the composition which comprises the ingredient a) of the invention is an aqueous composition which is at an acidic pH, in particular at a pH of between 1 and 6, more particularly between 2 and 5, preferably between 3 and 4, inclusive.

The pH values may be adjusted with an organic or mineral acid, or with an alkaline agent chosen from mineral or organic or hybrid alkaline agents or mixtures thereof.

The term “organic acid” is intended to mean an acid, i.e. a compound that is capable of releasing a cation or proton H⁺or H₃O⁺, in aqueous media, which comprises at least one optionally unsaturated, linear or branched C₁-C₂₀ hydrocarbon-based chain, a (hetero)cycloalkyl or (hetero)aryl group and at least one acidic chemical function chosen in particular from carboxyl C(O)OH, sulfuric SO₃H, SO₂H, and phosphoric PO₃H₂, PO₄H₂.

More particularly, the acids used are chosen from hydrochloric acid HCl, hydrobromic acid HBr, sulfuric acid H₂SO₄, alkylsulfonic acids: (C₁-C₆)Alk-S(O)₂OH such as methylsulfonic acid and ethylsulfonic acid; arylsulfonic acids: Ar—S(O)₂OH such as benzenesulfonic acid and toluenesulfonic acid; (C₁-C₆)alkoxysulfinic acids: Alk-O—S(O)OH such as methoxysulfinic acid and ethoxysulfinic acid; aryloxysulfinic acids such as tolueneoxysulfinic acid and phenoxysulfinic acid; phosphoric acid H₃PO₄; triflic acid CF₃SO₃H and tetrafluoroboric acid HBF₄, and carboxylic acid(s) of formula (II) below:

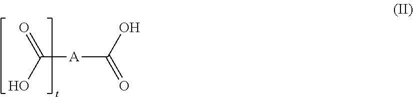

in which formula (II) or a salt thereof:

A represents a saturated or unsaturated, cyclic or non-cyclic, and aromatic or non-aromatic hydrocarbon-based group, which is monovalent when t is 0 or polyvalent when t is greater than or equal to 1, comprising from 1 to 50 carbon atoms, which is optionally interrupted with one or more heteroatoms and/or optionally substituted, especially with one or more hydroxyl groups; preferably, A represents a monovalent (C₁-C₆)alkyl group or a polyvalent (C₁-C₆)alkylene group optionally substituted with one or more hydroxyl groups.

Particularly, the carboxylic acids of formula (II) as defined above, and preferably the acid(s) used, is (are) an alpha-hydroxy acid such as lactic acids, glycolic acids, tartaric acids or citric acids.

The mineral alkaline agent(s) are preferably chosen from aqueous ammonia, alkaline carbonates or bicarbonates such as sodium or potassium carbonates and sodium or potassium bicarbonates, sodium hydroxide or potassium hydroxide, or mixtures thereof.

According to an advantageous embodiment of the invention, the alkaline agent(s) are organic amines, i.e. they contain at least one substituted or unsubstituted amino group.

The organic alkaline agent(s) are more preferentially chosen from organic amines with a pK_{b} at 25° C. of less than 12, preferably of less than 10 and even more advantageously of less than 6. It should be noted that it is the pK_{b} corresponding to the function of highest basicity.

Hybrid compounds that may be mentioned include the salts of the amines mentioned previously with acids such as carbonic acid or hydrochloric acid.

The organic alkaline agent(s) are chosen, for example, from alkanolamines, oxyethylenated and/or oxypropylenated ethylenediamines, amino acids and the compounds of formula (III) below:

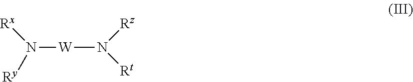

in which formula (III):
W is a divalent C₁-C₆ alkylene radical optionally substituted with a hydroxyl group or a C₁-C₆ alkyl radical, and/or optionally interrupted with one or more heteroatoms such as oxygen or NR^{u};
R^{x}, R^{y}, R^{z}, R^{t} and R^{u}, which may be identical or different, represent a hydrogen atom or a C₁-C₆ alkyl, C₁-C₆ hydroxyalkyl or C₁-C₆ aminoalkyl radical.

Preferably, the alkanolamine is ethanolamine (or monoethanolamine).

In one variant of the invention, the composition comprises, as alkaline agent, one or more alkanolamines (preferably ethanolamine) and aqueous ammonia. In this variant, the alkanolamine(s) are present in a predominant amount relative to the aqueous ammonia.

More preferentially, the pH is adjusted so that the pH is between 2.5 and 9.5 inclusive, or between 1 and 6 inclusive, more particularly between 2 and 5 and preferably between 3 and 4, by means of NH₄OH or citrate buffer.

### The Process For Treating Keratin Materials

The process for treating keratin materials, in particular keratin fibers, more particularly human keratin fibers such as the hair, consists, during a first step, in applying, to said materials, a composition comprising one or more ingredients a) as defined previously.

According to one particular embodiment of the invention, the process is carried out at ambient temperature (25° C.) without heat treatment of said keratin materials.

According to another particular embodiment of the invention, the process for treating keratin materials comprises, after the application of the composition comprising a), an additional step of heating the keratin materials, in particular keratin fibers, preferably to a temperature of at least 100° C., in particular to a temperature of between 100° C. and 250° C. inclusive. Preferably, the step of heating the keratin fibers is carried out at a temperature ranging from 150° C. to 220° C., preferably ranging from 160° C. to 220° C., preferentially ranging from 160° C. to 200° C., especially ranging from 170° C. to 190° C. It is understood that the heating temperatures are the temperatures of the heating means, in particular the iron plates when it is an iron, and not the temperature of said keratin materials.

This heating step b) is advantageously carried out by means of an iron.

The heating step makes it possible to optimize the effects of the process, and especially to optimize the persistence of the cosmetic properties after one or more shampooing washes.

For the purposes of the present invention, the term “iron” is intended to mean a device for heating keratin fibers by placing said fibers in contact with the heating device.

The end of the iron which comes into contact with the keratin materials, in particular keratin fibers, generally has two flat surfaces. These two surfaces may be made of metal or of ceramic. In particular, these two surfaces may be smooth or crimped or curved.

The heating step can be carried out using a straightening iron, a curling iron, a crimping iron or a steam iron. Preferably, the heating step is carried out using a straightening iron.

As examples of irons that may be used in the heating process according to the invention, mention may be made of any type of flat iron, and in particular, in a nonlimiting manner, those described in patents U.S. Pat. Nos. 5,957,140 and 5,046,516.

The iron may be applied by successive separate strokes lasting a few seconds or by gradual movement or sliding along the locks of keratin fibers, especially of hair.

Preferably, the iron is applied in the process according to the invention by a continuous movement from the root to the end of the hair, in one or more passes, in particular in two to twenty passes. The duration of each pass of the iron may range from 2 seconds to 1 minute.

Preferably, the step of heating the keratin fibers is performed for a time that may range from 2 seconds to 30 minutes, and preferentially from 2 seconds to 20 minutes, better still from 2 seconds to 10 minutes, better still from 2 seconds to 5 minutes and even better still from 2 seconds to 2 minutes.

The process according to the invention may also comprise an additional step c) of drying the keratin fibers after the application a) of the oxidized amino polysaccharide(s) and before step b) of heating the keratin fibers, carried out at a temperature of at least 100° C., if there is a heating step.

The drying step may be performed using a hairdryer or a drying hood or by drying in the open air. The drying step is advantageously carried out at a temperature ranging from 20° C. to 70° C.

After the step of applying the ingredients a) or the heating step b), the keratin fibers can be optionally rinsed with water or washed with a shampoo. The keratin fibers are subsequently optionally dried using a hairdryer or a drying hood or naturally.

According to one embodiment, the process according to the invention is performed on natural keratin fibers, especially natural hair.

According to another embodiment, the process according to the invention is carried out on damaged keratin fibers, especially damaged hair. As indicated previously, the term “damaged hair” is intended to mean dry or coarse or brittle or split or limp hair.

According to another embodiment, the treatment process according to the invention is preferably carried out on sensitized keratin fibers, especially sensitized hair, such as bleached, artificially dyed, relaxed or permanent-waved fibers.

The process according to the invention may be performed on keratin fibers, especially hair, which is wet or dry. Preferentially, the process is performed on dry keratin fibers, especially dry hair.

The composition(s) used according to the invention are advantageously applied to the keratin fibers in an amount ranging from 0.1 to 10 grams and preferably from 0.2 to 5 grams of composition per gram of keratin fibers.

After application of the composition to the keratin materials, in particular the keratin fibers, they may be wrung out to remove the excess composition or washed with water or with a shampoo.

After application to the keratin materials, in particular the keratin fibers, of the ingredients a) as defined previously, and before carrying out b) the step of heating said keratin fibers, if there is a heating step, under one particular embodiment of the invention a) the oxidized amino polysaccharide(s) a) as defined previously are left on for a period of time ranging from 1 to 60 minutes, preferably ranging from 2 to 50 minutes, preferentially ranging from 5 to 45 minutes. The leave-on period may take place at a temperature ranging from 15° C. to 45° C., preferably at ambient temperature (25° C.).

According to one embodiment of the process according to the invention, the oxidized amino polysaccharide(s) comprising at least one carboxy(late) group a) as defined previously and the (poly)saccharide(s) comprising amine group(s) b) as defined previously are present in separate compositions. They are therefore applied separately to said keratin materials.

According to one particular embodiment of the process of the invention, the step of applying the oxidized polysaccharide(s) comprising at least one carboxy(late) group a) employs a composition comprising at least one oxidized amino polysaccharide comprising at least one carboxy(late) group as defined previously, in a content ranging from 0.05% to 15% by weight, relative to the total weight of the composition, preferably ranging from 0.1% to 10% by weight, more preferentially ranging from 0.2% to 6% by weight.

According to one embodiment of the process according to the invention, the following steps are carried out, in the following order: the step of applying the ingredient a) and then the heating step b). Advantageously, the ingredients a) are present in a cosmetic composition.

The treatment process according to the invention may be performed before, during and/or after an additional process of cosmetic treatment of the keratin fibers, such as a process for temporarily shaping (shaping with curlers, a crimping iron or a straightening iron) or a process for durably shaping (permanent-waving or relaxing) the keratin fibers.

The treatment process may be performed as a pre-treatment to a dyeing or relaxing process and/or a permanent-waving process so as to cosmetically protect the keratin fibers against these treatments. In other words, this process is performed to preserve the cosmetic properties of the keratin fibers before a cosmetic treatment process as described previously.

In particular, the treatment process is performed as a post-treatment to a bleaching, artificial dyeing or relaxing process and/or a permanent-waving process so as to repair said fibers.

The process according to the invention may be performed during a cosmetic treatment process so as to repair said fibers.

In particular, the treatment process according to the invention may be carried out on damaged keratin fibers.

In other words, the treatment process according to the invention is preferably carried out on sensitized keratin fibers such as bleached, dyed, relaxed or permanent-waved fibers.

In particular, the treatment process may be carried out before a bleaching, dyeing or relaxing process and/or a permanent-waving process on keratin fibers.

As a variant, the treatment process may be carried out during and/or after a cosmetic process for treating keratin fibers, in particular:

i) during and/or after a process for dyeing or a process for permanent-waving or a process for relaxing keratin fibers, and

ii) after a process for bleaching keratin fibers.

According to one embodiment, the treatment process according to the invention is performed after a process of bleaching the keratin fibers.

### The Kit

A subject of the invention is also a multi-compartment kit or device comprising:
a first composition comprising a) at least one oxidized amino polysaccharide comprising at least one carboxy(late) group as defined previously; and
optionally, a device for heating the keratin fibers to a temperature of at least 100° C., preferably ranging from 100 to 250° C.

The composition packaging assembly is, in a known manner, any packaging that is suitable for storing cosmetic compositions (in particular a bottle, tube, spray bottle or aerosol bottle).

Such a kit allows the process for treating keratin fibers according to the invention to be performed.

The examples that follow are given as illustrations of the present invention.

The amounts indicated in the examples are expressed as weight percentages.

### EXAMPLES

The following compositions were prepared; the % are percentages by weight in g per 100 g of composition.

| Compositions | | Ingredients |
|---|---|---|
| Composition 1 | Control | Milli-Q water only |
| Composition 2 | Invention | Oxidized and carboxylated amino |
| | | inulin* at 5% in water, spontaneous |
| | | pH 5.5 |
| Composition 3 | Comparative | Oxidized inulin** at 5% in water, |
| | | spontaneous pH 3 |

**The oxidized inulin (OI) was prepared by oxidation of inulin sold under the name Inutec N25 by Orafti, by performing a reactive extrusion process as described in the article “Water soluble oxidized starches by peroxide reactive extrusion” by R. E. Wing and J. L. Willett, Industrial Crops and Products 7, 1997, pages 45-52. A BC21 co-rotating twin-screw extruder sold by the company Clextral was used, and aqueous hydrogen peroxide solution was used as oxidizing agent. OI is thus obtained by reactive extrusion of a mixture of 78% by weight of inulin and 1.57% by weight of aqueous hydrogen peroxide solution; the spontaneous pH after reactive extrusion is 3.8. The OI thus obtained has a carbonyl content of 1.23% (w/w) and a carboxyl content of 0.17% (w/w).
*the oxidized and carboxylated amino inulin according to the invention is obtained from the OI which was aminated with aqueous ammonia, reduced with palladium on graphite in the presence of hydrogen under pressure (20 bar) and heated at 50° C. for several hours (between 10 h and 24 h), followed by elimination of the Pd/C, of nitrogen and of the water; the amination step was carried out according to the conventional technique of reductive amination in the form of ammonium salt of inulin from (cf. Chem. Eur. J., 1130-1144 (2007). ibid 14. 10196 (2008)).

The physicochemical analyses are in agreement with the presence of amino, carbonyl and carboxy groups in the inulin of the invention.

### Treatment

### Evaluation Protocol

The tests for evaluating the treatment of the keratin fibers were carried out in the following way:

20 cm locks of straight Caucasian hair, weighing 2.7 g, were bleached with 2 INFINIE PLATINE bleachings, rinsed and dried and then locks of dry hair were brought into contact with one of the compositions 1 to 3 in a proportion of 2 g of composition per 1 g of hair, 30 minutes of immersion at 55° C.; followed:
by wringing out the locks then drying with a hair dryer at 60° C. with passage of a soft brush,
by applying a straightening iron at 210° C.: 15 passes of 5 seconds;
by wetting the locks with tap water, applying a DOP chamomile shampoo in a proportion of 0.4 g/g of hair, massaging for 15 seconds and careful rinsing with water at 37° C. for 10 seconds; then
drying in an incubator at 60° C. for 10 minutes.

### Evaluations And Tests Carried Out

Feel: After applying and drying, the dry locks were evaluated sensorially on tactile criteria by 5 individuals who had been trained in the sensory evaluation of hair. A score from 1 (locks very coarse to the touch) to 5 (locks very soft to the touch) was given. The averages of these scores are reported.

Disentangling: Tests of disentangling using a comb were carried out after immersion for 10 seconds in water, by 5 passes of a small-toothed plastic comb (7 teeth/cm, diameter of the teeth approx. 800 μm). A score from 1 (locks very difficult to disentangle) to 5 (locks very easy to disentangle) was given. The averages of these scores are reported.

Visual smoothing: An evaluation of the visual smoothing is carried out on the dry appearance of the treated locks after 48 hours having been kept at 55% RH. A score from 1 (not smooth) to 5 (smooth locks) was given. The averages of these scores are reported.

### Results

The locks were evaluated by 5 testers. The following results were obtained (1: lowest score to 5: highest score)

| | | | Dry visual | |
|---|---|---|---|---|
| Compositions | Wet | Dry | smoothing | |
| tested | disentangling | disentangling | (manageability) | Dry feel |
| Composition 1 | 1.3 | 1.1 | 1.4 | 1.3 |
| (Placebo) | | | | |
| Composition 2 | 3.9 | 3.8 | 4.7 | 3.8 |
| (invention) | | | | |
| Composition 3 | 1.8 | 2.1 | 2.9 | 3.2 |
| (comparative) | | | | |

It appears that the process for treating keratin materials according to the invention and the composition of the invention employed in the process make it possible to significantly improve the disentangling, the smoothing and the feel of said materials.

1. A process for treating keratin materials, in particular keratin fibers, especially human keratin fibers, preferably the hair, comprising at least one step consisting in applying, to said fibers, a) one or more oxidized amino polysaccharide(s) comprising at least one carboxy group —C(O)—OH or carboxylate group —C(O)O⁻, M⁺with M⁺representing a cationic counterion.

2. The process as claimed in the preceding claim, wherein the polysaccharide(s) a) are nonionic or anionic.

3. The process as claimed in either one of the preceding claims, wherein said oxidized polysaccharide(s) a) are represented by formula (I) below:
(R₁R₂N)ₚ—P—(CHO)ₘ(COOX)ₙ (I)
in which formula (I):
P represents a polysaccharide chain constituted of monosaccharides comprising 5 carbon atoms or more than 5 carbon atoms, preferably 6 or more than 6 carbon atoms and more particularly 6 carbon atoms;
R₁ and R₂, which may be identical or different, represent i) a hydrogen atom, ii) a (C₁-C₁₄)alkyl group that is optionally substituted, preferably with one or more hydroxyl or NH₂ groups, iii) a (hetero)aryl group such as phenyl, iv) a (hetero)aryl(C₁-C₁₄)alkyl group such as benzyl, v) a (hetero)cyclo(C₅-C₇)alkyl group such as cyclohexyl, morpholinyl, piperazinyl, piperidinyl, vi) a (hetero)cyclo(C₅-C₇)alkyl(C₁-C₁₄)alkyl group such as cyclohexylmethyl, vii) —C(Y)—(Y′)—R′₁ with Y and Y′, which may be identical or different, representing an oxygen atom, a sulfur atom or N(R′₂), preferably oxygen, q=0 or 1, preferably 0; and R′₁ and R′₂ representing i) to vi) of R₁ and R₂ defined previously;
X represents hydrogen atom, a cationic counterion, in particular chosen from i) alkali metal, ii) alkaline earth metal, such as sodium or potassium, iii) ammonium R,R′,R″N⁺— with R, R′ and R″, which may be identical or different, representing a hydrogen ion, a (C₁-C₆)alkyl group optionally substituted in particular with a hydroxyl group, such as hydroxyethyl; iv) and a cationic counterion derived from aqueous ammonia, monoethanolamine, diethanolamine, triethanolamine and 3-amino-1,2-propanediol, and basic amino acids such as lysine, arginine, sarcosine, ornithine and citrulline;
m+n is greater than or equal to 1;
m is such that the degree of substitution of the polysaccharide with one or more aldehyde groups (DS(CHO)), which is greater than or equal to 0, in particular m is within the range of from 0 to 2, more particularly ranging from 0.001 to 1.8, preferably ranging from 0.005 to 1.5;
n is such that the degree of substitution of the polysaccharide with one or more carboxylic groups (DS(COOX)) is greater than 0, in particular less than or equal to 2, preferably between 0.001 and 1.5, inclusive;
p is such that the degree of substitution of the polysaccharide with one or more amine groups (DS(R₁R₂N)) is greater than 0, in particular less than or equal to 2, preferably between 0.001 and 1.5, inclusive;
preferably, m is within the range of from 0 to 1, n and p, which may be identical or different, are within the range of from 0.01 to 2.

4. The process as claimed in the preceding claim, characterized in that in particular R₁ and R₂ are chosen from a hydrogen atom and —C(O)—R′₁ representing i) to vi) as in the preceding claim; in particular R₁ and R₂ represent i) a hydrogen atom, ii) a (C₁-C₆)alkyl group such as methyl, or a (C₁-C₆)alkylcarbonyl group such as acetyl; preferably, R₁ and R₂ represent a hydrogen atom.

5. The process as claimed in claim 3 or 4, characterized in that the polysaccharide chain is chosen from celluloses, starches, guar gums, inulins, xanthan gums, pullulan gums, agar-agar gums, carrageenan gums, gellan gums, gums arabic, tragacanth gums, xylans and derivatives thereof, cellobiose, maltodextrin, scleroglucan, chitosan, ulvan, fucoidan, alginate, pectin, heparin, hyaluronic acid, and cyclodextrin and derivatives thereof, and mixtures thereof.

6. The process as claimed in claim 3 or 4, characterized in that the polysaccharide chain is chosen from celluloses, (C₁-C₆)alkylcelluloses, hydroxy(C₁-C₆)alkylcelluloses such as hydroxyethylcelluloses and hydroxypropylcelluloses, carboxycelluloses and carboxy(C₁-C₆)alkylcelluloses such as carboxymethylcelluloses, starches, inulins, and preferably inulin.

7. The process as claimed in any one of the preceding claims, wherein the step of applying the oxidized amino polysaccharide(s) comprising at least one carboxy(late) group a) employs a composition comprising at least one oxidized amino polysaccharide comprising at least one carboxylate group as defined in any one of the preceding claims, in a content ranging from 0.05% to 15% by weight, relative to the total weight of the composition, in particular ranging from 0.1% to 10% by weight, more preferentially ranging from 0.2% to 6% by weight.

8. The process as claimed in any one of the preceding claims, which employs a composition comprising at least one oxidized amino polysaccharide comprising at least one carboxylate group as defined in any one of the preceding claims, said composition being aqueous and at a pH of between 1 and 9.5, inclusive; in particular, said composition is at acidic pH, in addition particularly between 2 and 6, inclusive, even more particularly between 2.5 and 5, preferably between 3 and 4.

9. The process as claimed in any one of the preceding claims, which employs a composition comprising at least one oxidized amino polysaccharide comprising at least one carboxylate group as defined in any one of claims 1 to 7, said composition also comprising one or more additional ingredients chosen from nonionic, anionic, cationic and amphoteric surfactants, vitamins and provitamins, including panthenol, sunscreens, fillers, colorants, nacreous agents, opacifiers, sequestrants, film-forming polymers, cationic, anionic or neutral polymers other than the polysaccharide(s) a), associative polymers, plasticizers, thickeners, oils, antioxidants, antifoams, moisturizers, emollients, penetrants, fragrances and preservatives; preferably, the composition also comprises one or more nonionic, anionic, cationic or amphoteric surfactants, cationic, anionic or neutral polymers other than the polysaccharides a), or associative polymers.

10. The process as claimed in any one of the preceding claims, which comprises an additional step of heating which is carried out at a temperature ranging from 100° C. to 250° C., preferably ranging from 150° C. to 220° C., preferentially ranging from 160° C. to 220° C., more preferentially ranging from 160° C. to 200° C.; preferably, this step takes place after the application of the oxidized amino polysaccharide(s) comprising at least one carboxy(late) group a); particularly, the heating step is carried out with a straightening iron.

11. An aqueous composition at a pH of between 1 and 9.5, inclusive, in particular at acidic pH, in addition particularly between 2 and 6, inclusive, even more particularly between 2.5 and 5, preferably between 3 and 4, comprising one or more oxidized amino polysaccharide(s) comprising at least one carboxy(late) a) as defined in any one of claims 1 to 7 and 9.

12. The use of one or more oxidized amino polysaccharide(s) comprising at least one carboxy(late) a) as defined in any one of claims 1 to 6, for improving the conditioning of keratin fibers and/or for making manageable and/or for repairing damaged keratin fibers and/or for preventing breakage of keratin fibers.

13. The use of a composition as defined in claim 11, for improving the conditioning of keratin fibers and/or for making manageable and/or for repairing damaged keratin fibers, and/or for preventing breakage of keratin fibers

14. A multi-compartment kit or device comprising:
a first composition as defined in claim 11; and
optionally, a device for heating the keratin fibers to a temperature of at least 100° C., preferably ranging from 100 to 250° C.
